(19) United States
(12) Patent Application Publication
(10) Pub. No.: US 2016/0184047 A1
(43) Pub. Date: Jun. 30, 2016

(54) Surgical Site Indicator

(71) Applicant: Marquis Weir, Memphis, TN (US)

(72) Inventor: Marquis Weir, Memphis, TN (US)

(21) Appl. No.: 14/885,439

(22) Filed: Oct. 16, 2015

Related U.S. Application Data
(60) Provisional application No. 62/096,626, filed on Dec. 24, 2014.

(51) Int. Cl. A61B 90/00; A61B 90/94

(52) U.S. Cl. CPC A61B 90/39; A61B 90/94; A61B 2090/3937

(57) ABSTRACT

A surgical site indicator for use in marking the location on a patient's body where a surgery is to be conducted. The surgical site indicator includes a bandage having an upper surface and a lower surface. The upper surface includes indicia thereon to indicate that the bandage marks a surgical site. The lower surface includes an adhesive for securing the surgical site indicator onto a patient's body in a desired position. Further, the lower surface includes a marking substance that leaves an imprint or mark on the patient's body so that if the bandage is removed, the marking remains on the body part of the patient where the surgery will be conducted. In this way, the surgical site indicator helps to remind healthcare professionals of the surgery location to avoid any confusion and to help prevent mistakes from occurring.

### CROSS REFERENCE TO RELATED APPLICATION

This application claims the benefit of U.S. Provisional Application Ser. No. 62/096,626 filed on Dec. 24, 2014. The above identified patent application is herein incorporated by reference in its entirety to provide continuity of disclosure.

### BACKGROUND OF THE INVENTION

1. Field of the Invention

The present invention relates to marking devices. More specifically, the present invention provides a surgical site indicator that helps healthcare professionals to easily determine what part of a patient's body on which to operate. The surgical site indicator comprises a bandage having an upper surface with indicia thereon, and a lower surface having an adhesive and a marking substance thereon that is designed to leave an imprint on the patient's body.

Healthcare professionals often mark the body part of a patient where a surgery is scheduled to be performed. This helps to prevent the healthcare professionals from mistakenly operating on the wrong body part of a patient. Mistakes are particularly likely to be made when a surgery is performed on one part of the user's body, such as the user's left leg, wherein the healthcare professional may accidentally begin to operate on the user's right leg. Such mistakes are detrimental to the patient, and may result in delay or cancellation of the surgery. Further, the healthcare professionals may be involved in malpractice cases for operating on the wrong body part of the patient.

In order to mark a patient's body for surgery, a healthcare professional may simply make a slash, X, or other mark with a marker. However, the use of a marker does not convey any information to other healthcare professionals. Another healthcare professional may not realize the mark has been made or may not appreciate the significance of the mark. Further, it can be unclear who made the mark, and it can be unclear if the mark accurately identifies the location for an operation. Thus, an improved surgical site indicator is desired in order to help medical professionals identify the site of a surgery.

Devices have been disclosed in the prior art that relate to surgical site marking systems. These include devices that have been patented and published in patent application publications. These devices generally relate to labels or bandages having markings or indicia thereon, such as U.S. Published Patent Application Number 2004/0056477, U.S. Published Patent Application Number 2003/0182815, U.S. Published Patent Application Number 2006/0266368, U.S. Published Patent Application Number 2002/0040202, and U.S. Published Patent Application Number 2013/0043669.

These prior art devices have several known drawbacks. While the devices in the prior art include labels or bandages for marking or indicating a surgical site. These devices, however, fail to provide a device that is designed to leave an imprint or mark on the user's body and instead require a bandage or label to be adhesively affixed to the user's body. Such bandages may fall off, causing the surgical site to no longer be properly marked.

In light of the devices disclosed in the prior art, it is submitted that the present invention substantially diverges in design elements from the prior art and consequently it is clear that there is a need in the art for an improvement to existing surgical site indicator devices. In this regard the instant invention substantially fulfills these needs.

### SUMMARY OF THE INVENTION

In view of the foregoing disadvantages inherent in the known types of surgical site indicators now present in the prior art, the present invention provides a new surgical site indicator wherein the same can be utilized for providing convenience for the user when marking the location of a surgery on a patient's body.

It is therefore an object of the present invention to provide a new and improved surgical site indicator comprising a bandage having an upper surface with indicia thereon and a lower surface with an adhesive and a marking substance adapted to leave an imprint on a patient's skin.

It is another object of the present invention to provide a surgical site indicator wherein the upper surface thereof can be written on or marked.

Another object of the present invention is to provide a surgical site indicator that can be used on any portion of a patient's body.

Yet another object of the present invention is to provide a surgical site indicator composed of a flexible material.

Another object of the present invention is to provide a surgical site indicator that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front view of the surgical site indicator of the present invention.

FIG. 2 shows a rear view of the surgical site indicator of the present invention.

FIG. 3 shows a perspective view of the surgical site indicator in use.

### DETAILED DESCRIPTION OF THE INVENTION

Reference is made herein to the attached drawings. Like reference numerals are used throughout the drawings to depict like or similar elements of the surgical site indicator. For the purposes of presenting a brief and clear description of the present invention, the preferred embodiment will be discussed as used for marking the location of a surgical site so as to allow healthcare professionals to easily determine the location of a surgical site. The figures are intended for representative purposes only and should not be considered to be limiting in any respect.

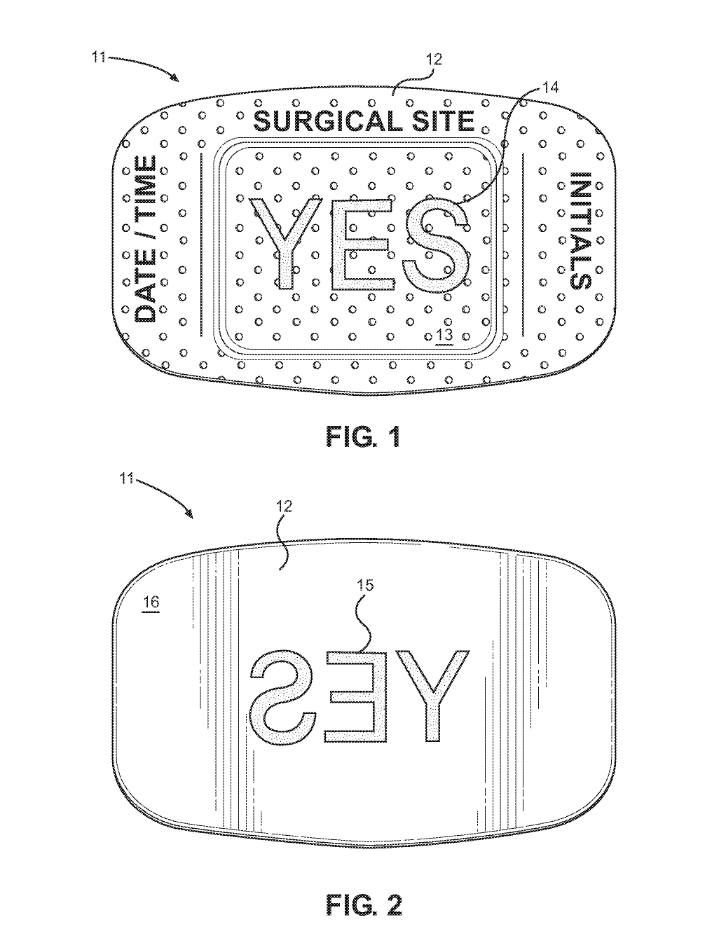

Referring now to FIGS. 1 and 2, there is shown a front view of the surgical site indicator and a rear view thereof, respectively. The surgical site indicator 11 comprises a bandage 12 having an upper surface 13 and a lower surface 16. The upper surface 13 includes one or more indicia 14 thereon for marking the surgical site. The indicia 14 may include a word or phrase, such as the word “yes” for indicating that the surgeon should operate on the marked site. However, various other words, phrases, or indicia may be used, such as a letter “X” to mark the location of the surgical site.

The upper surface of the bandage 12 is composed of a material that is adapted to be written on using a pencil, pen, marker, or other writing instrument. In this way, a healthcare professional can mark the date or time of the surgery on the upper surface 13 of the surgical site indicator 11. Further, the healthcare professional can write his or her initials thereon so that others may know who placed the surgical site indicator onto a patient.

The lower surface 16 is coextensive with the upper surface 13 and the lower surface 16 is adapted to be placed directly against a user's body. The bandage 12 is composed of a flexible material so that the bandage 12 can contour to a user's body part, such as a knee, elbow, or various other portions of a patient's body. In some embodiments, the lower surface 16 comprises an adhesive thereon for temporarily affixing the bandage 12 to the patient's body. Preferably, the bandage 12 includes a pressure sensitive adhesive. The adhesive may be disposed on the entirety of the lower surface 16 or may be disposed only on a portion thereof.

Further, the lower surface 16 comprises a marking substance 15 that defines indicia similar to or the same as the indicia 14 on the upper surface 13 of the bandage 12. In the illustrated embodiment, the lower surface 16 comprises the word “YES” wherein the word is created using a marking substance 15, such as a dye. The marking substance 15 is adapted to leave an imprint on the patient's body when the marking substance 15 is brought into contact therewith. The marking substance 15 is a nontoxic, water soluble dye or other similar material. Further, the dye dries on contact with skin so that the dye does not rub off onto other surfaces or materials.

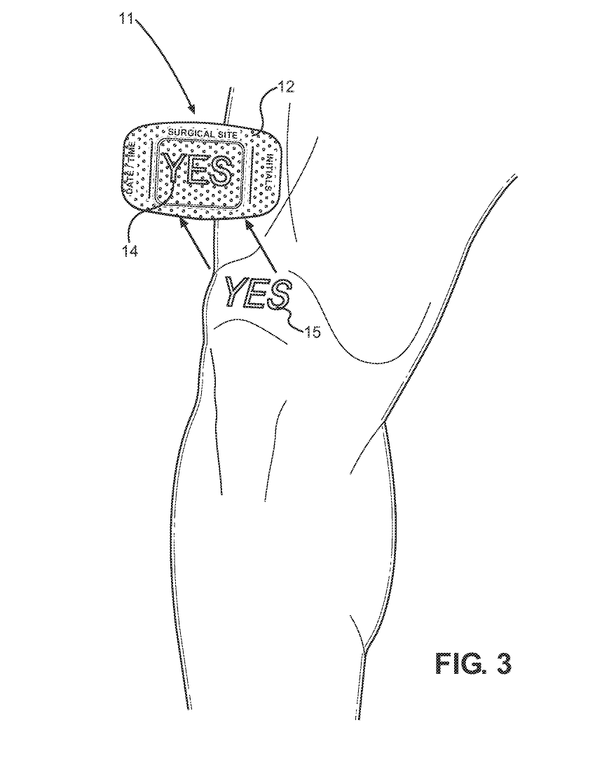

Referring now to FIG. 3, there is shown a perspective view of the surgical site indicator in use. In operation, a healthcare professional can secure the surgical site indicator 11 onto the user's body by placing the lower surface in contact with the user's body. The adhesive on the lower surface serves to removably secure the bandage 12 onto the patient's body in the desired location.

Once the surgical site indicator 11 is affixed to the user's body, the marking substance 15 thereon is brought into contact with the user's body, wherein the marking substance 15 will leave a mark or imprint on the user's body. The marking substance 15 marks the skin of the patient so that even if the surgical site indicator 11 is removed, the surgical site will be still marked. Preferably, the user's skin is marked with a word or other indicia 14 indicating the location of the surgical site, such as an X, the word YES, among various other indicia 14. The bandage 12 can then be removed, and the patient's body part will bear a mark created by the marking substance 15 on the surgical site indicator 11.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A surgical site indicator, comprising:
a bandage having an upper surface and a lower surface;
wherein said lower surface comprises an adhesive thereon for affixing said bandage to a user's skin;
wherein said lower surface further comprises a marking substance adapted to create a mark on said user's skin when pressed thereagainst such that said mark remains on said user's skin when said bandage is removed therefrom.

2) The surgical site indicator of claim 1, wherein said bandage is composed of a flexible material adapted to contour to a body part of a user when applied thereto.

3) The surgical site indicator of claim 1, wherein said marking substance comprises a dye.

4) The surgical site indicator of claim 1, wherein said upper surface comprises indicia thereon, wherein said indicia is adapted to indicate a surgical site.

5) The surgical site indicator of claim 4, wherein said indicia comprises a word or phrase.

6) The surgical site indicator of claim 1, wherein said upper surface of said bandage is composed of a material adapted to be written on by a writing instrument.
